(19) United States
(12) Patent Application Publication
(10) Pub. No.: US 2007/0082952 A1
(43) Pub. Date: Apr. 12, 2007

(54) Pharmaceutical compositions, methods of preparation thereof, and methods of treatment

(76) Inventor: Eric J. Benjamin, Jamestown, NC (US)

Correspondence Address: KILPATRICK STOCKTON LLP - 41305;CHARLES CALKINS, 1001 WEST FOURTH STREET, WINSTON-SALEM, NC, 27101, US

(21) Appl. No.: 11/544,362

(22) Filed: Oct. 6, 2006

Related U.S. Application Data
(60) Provisional application No. 60/758,740, filed on Jan. 13, 2006.
(60) Provisional application No. 60/756,287, filed on Jan. 5, 2006.
(60) Provisional application No. 60/724,010, filed on Oct. 6, 2005.

(51) Int. Cl. A61K 31/195

(52) U.S. Cl. 514/563; 562/450

(57) ABSTRACT

The present invention provides compositions, useful as pharmaceuticals, comprising 3-biphenyl-4-yl-(2S)-[(4′-trifluoromethyl-biphenyl-4-carbonyl)-amino]-propionic acid. Also disclosed are methods for preparing the compositions and methods for using the compositions.

The present invention claims priority under 35 U.S.C. 119(e) to U.S. Provisional Applications 60/758,740 filed Jan. 13, 2006, 60/756,287 filed Jan. 5, 2006, and 60/724,010 filed Oct. 6, 2005, all of which are incorporated herein by reference in their entireties.

### FIELD OF THE INVENTION

The present invention relates to compositions comprising a compound that may be used to inhibit thrombosis, methods of preparing the compositions; and methods of using the compositions.

### BACKGROUND OF THE INVENTION

Hemostasis, the arrest of bleeding from an injured blood vessel, generally necessitates the concerted activity of vascular, platelet, and/or plasma factors to eventually form a hemostatic seal or a blood clot.

Many significant cardiovascular disease states are related to abnormal hemostasis. With respect to the coronary arterial vasculature, abnormal thrombus formation due to the rupture of an established atherosclerotic plaque is a major cause of acute myocardial infarction and unstable angina. Moreover, treatment of an occlusive coronary thrombus by either thrombolytic therapy or percutaneous transluminal coronary angioplasty (PTCA) is often accompanied by an acute thrombotic re-closure of the affected vessel which requires immediate resolution. With respect to the venous vasculature, a high percentage of patients undergoing major surgery in the lower extremities or the abdominal area suffer from thrombus formation in the venous vasculature which can result in reduced blood flow to the affected extremity and a predisposition to pulmonary embolism. Disseminated intravascular coagulopathy commonly occurs within both vascular systems during septic shock, certain viral infections and cancer and is characterized by the rapid consumption of coagulation factors and systemic coagulation which results in the formation of life-threatening thrombi occurring throughout the vasculature leading to widespread organ failure.

### SUMMARY OF THE INVENTION

Compounds such as those described in US Patent Publication No. 20040110832, published Jun. 10, 2004, the disclosure of which is hereby incorporated herein by reference, may be used to inhibit clotting in the intrinsic clotting pathway and may be useful in the treatment of various cardiovascular diseases by modifying thrombus formation and growth. 3-Biphenyl-4-yl-(2S)-[(4′-trifluoromethyl-biphenyl-4-carbonyl)-amino]-propionic acid is disclosed and described in US Patent Publication No. 20040110832 and may be used to inhibit clotting activity in the intrinsic clotting pathway, herein incorporated by reference in its entirety.

In an aspect, the present invention provides compositions comprising 3-biphenyl-4-yl-(2S)-[(4′-trifluoromethyl-biphenyl-4-carbonyl)-amino]-propionic acid.

In another aspect, the present invention provides methods and/or processes for producing compositions comprising 3-biphenyl-4-yl-(2S)-[(4′-trifluoromethyl-biphenyl-4-carbonyl)-amino]-propionic acid.

In a further aspect, the present invention provides methods for using compositions comprising 3-biphenyl-4-yl-(2S)-[(4′-trifluoromethyl-biphenyl-4-carbonyl)-amino]-propionic acid. The compositions comprising 3-biphenyl-4-yl-(2S)-[(4′-trifluoromethyl-biphenyl-4-carbonyl)-amino]-propionic acid may be used for the treatment of a variety of applications including management, treatment, control of diseases or conditions in humans. Such diseases or disease states include cardiovascular diseases, atrial fibrillation, cardiopulmonary bypass, stroke, myocardial infarction, deep vein thrombosis associated with surgical procedures or long periods of confinement, acute and chronic inflammation and clotting associated with hemodialysis. Further, the compositions of the present invention may also be useful for the treatment of diseases or conditions caused in part by the intrinsic clotting pathway using Factor IX.

### DETAILED DESCRIPTION OF THE INVENTION

For the purposes of this specification, unless otherwise indicated, all numbers expressing quantities of ingredients, reaction conditions, and so forth used in the specification are to be understood as being modified in all instances by the term “about.” Accordingly, unless indicated to the contrary, the numerical parameters set forth in the following specification are approximations that can vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g. 1 to 6.1, and ending with a maximum value of 10 or less, e.g., 5.5 to 10. Additionally, any reference referred to as being “incorporated herein” is to be understood as being incorporated in its entirety.

By percent by weight it is meant that a particular weight of one ingredient in a composition is divided by the total weight of all of the ingredients in that composition. Percent by weight may be used interchangeably and means approximately the same as weight/weight percent or % (weight/weight) or percent by mass or mass percent. When a liquid solute is used, it is often more practical to use volume/volume percent or % (vol/vol) or percent by volume, which are all considered to be synonymous. Ppm (parts per million), ppb (parts per billion), pph (parts per hundred) are often used to indicate a percentage based on quantity and not on mass (i.e., the quantity of a given type of atom or a given type of molecule in a composition with more atoms or molecules (be it gas, liquid or solid) is divided by the total quantity of atoms or molecules in the total composition). Other terms that are used are molarity, which is the number of moles of solute per liters of solution, molality, which is the number of moles of solution per kilograms of solution. Another concentration unit is the mole fraction, which is the moles of a given component divided by the total moles of all solution components. Mole percent is related to the mole fraction and is the mole fraction multiplied by 100.

It is further noted that, as used in this specification, the singular forms “a,” “an,” and “the” include plural referents unless expressly and unequivocally limited to one referent.

The term “factor IX” is used herein to refer to blood coagulation factor IX, including both activated and non-activated forms thereof.

The term “therapeutically effective amount” is used herein to denote that amount of 3-biphenyl-4-yl-(2S)-[(4′-trifluoromethyl-biphenyl-4-carbonyl)-amino]-propionic acid that will elicit the therapeutic response of a subject that is being sought. In an embodiment, the therapeutic response may be partial inhibition of the biological function of factor IX. In another embodiment, the therapeutic effective amount may be a sustained blood level of less than 1 μM. In another embodiment, the therapeutic effective amount may be a sustained blood level of greater than 0.1 μM.

The term “treatment” as used herein, refers to the full spectrum of treatments for a given condition or disorder from which a patient is suffering, including alleviation of one, most, or all of the symptoms resulting from that disorder, to the prevention of the onset of the disorder.

As used herein 3-biphenyl-4-yl-(2S)-[(4′-trifluoromethyl-biphenyl-4-carbonyl)-amino]-propionic acid refers to the composition disclosed and described in US Patent Publication No. 20040110832. In embodiments, the compositions and methods of the present invention may also use a pharmaceutically acceptable salt of 3-biphenyl-4-yl-(2S)-[(4′-trifluoromethyl-biphenyl-4-carbonyl)-amino]-propionic acid.

An obstacle to the use of 3-biphenyl-4-yl-(2S)-[(4′-trifluoromethyl-biphenyl-4-carbonyl)-amino]-propionic acid in certain pharmacological forms is its aqueous solubility (less than 0.5 micrograms/milliliter (μg/mL)), which may make the bioavailability of biphenyl-4-yl-(2S)-[(4′-trifluoromethyl-biphenyl-4-carbonyl)-amino]-propionic acid lower than desirable. A composition of the present invention, having an increased dissolution rate, advantageously enhances the bioavailability of 3-biphenyl-4-yl-(2S)-[(4′-trifluoromethyl-biphenyl-4-carbonyl)-amino]-propionic acid, particularly when dosed orally.

Another obstacle in formulating solid compositions of 3-biphenyl-4-yl-(2S)-[(4′-trifluoromethyl-biphenyl-4-carbonyl)-amino]-propionic acid is its fine particle size and its cohesive nature. These properties can result in poor flow, poor density and poor compression characteristics in a solid dose formulation making scale-up of the formulation difficult. In contrast, compositions of the present invention may advantageously exhibit improved flow and compression characteristics that simplify scale-up. The present invention provides a composition that may have improved density, flow, shear, and/or particle size.

In an embodiment, the present invention provides a composition comprising 3-biphenyl-4-yl-(2S)-[(4′-trifluoromethyl-biphenyl-4-carbonyl)-amino]-propionic acid and a water-soluble polymer. In another embodiment, the composition further comprises a surfactant. In another embodiment, the composition further comprises one or more of: a filler; a binder; a diluent; a glidant; a lubricant; disintegrant; or a similar ingredient that facilitates the granulation and tableting process.

In another embodiment the present invention provides a composition comprising 3-biphenyl-4-yl-(2S)-[(4′-trifluoromethyl-biphenyl-4-carbonyl)-amino]-propionic acid and a surfactant. In an embodiment, the composition further comprises a water-soluble polymer. In an embodiment the composition further comprises one or more of: a filler; a binder; a diluent; a glidant; a lubricant; disintegrant; or a similar ingredient that facilitates the granulation and tableting process.

Embodiments of a composition of the present invention may be advantageously utilized in tablet form to provide an oral dosage of 3-biphenyl-4-yl-(2S)-[(4′-trifluoromethyl-biphenyl-4-carbonyl)-amino]-propionic acid with increased bioavailability relative to compositions with lower rates of dissolution.

The pharmaceutical compositions of the present invention include the 3-biphenyl-4-yl-(2S)-[(4′-trifluoromethyl-biphenyl-4-carbonyl)-amino]-propionic acid primarily in a solid state. Further, the pharmaceutical compositions of the present invention may be in a solid dosage form where the active ingredient is in a solid state and the dosage may be a powder, sphere, capsule, or a tablet.

Water soluble polymers suitable for use in the present invention include a water soluble polymer that allows processing of the composition into a solid dosage form (e.g. tablet, pill, capsule) while improving the bioavailability of 3-biphenyl-4-yl-(2S)-[(4′-trifluoromethyl-biphenyl-4-carbonyl)-amino]-propionic acid. Water soluble polymers that may be suitable for use in the present invention include, but are not limited to, povidone (PVP), hydroxypropylmethylcellulose (HPMC), polyethylene glycol (PEG), cyclodextrins, and/or mixtures thereof. In an embodiment, the water soluble polymer is PVP having a molecular weight of between 1 and 1000 kilodaltons. In another embodiment, the water soluble polymer is PVP having a molecular weight between 2.5 and 100 kilodaltons. In a further embodiment, the water soluble polymer is PVP having a molecular weight between 30 and 70 kilodaltons. In a particular embodiment, the water soluble polymer is PVP having a molecular weight of 50 kilodaltons.

In an embodiment, the amount of water soluble polymer present in the composition is an amount sufficient to reduce the amount of fines produced when the wet granulation is dried. In one non-limiting example, the amount of the water soluble polymer PVP operable to reduce the amount of fines produced when drying the wet granulation is greater than about 1.5%, by weight of the composition. In various embodiments, the amount of the water soluble polymer PVP operable to reduce the amount of fines produced when drying the wet granulation may be greater than about 0.5% or may be greater than about 3.0%, by weight.

Surfactants suitable for use in the present invention include a surfactant that allows processing of the composition into a solid dosage form (e.g. tablet, pill, capsule) while enhancing the dissolution of 3-biphenyl-4-yl-(2S)-[(4′-trifluoromethyl-biphenyl-4-carbonyl)-amino]-propionic acid from solid form. Surfactants that may be suitable for use in the present invention, include, but are not limited to, polysorbate 80, sodium lauryl sulfate, sodium dodecyl sulfate, salts of bile acids (taurocholate, glycocholate, cholate, deoxycholate, etc.) which may be combined with lecithin; ethoxylated vegetable oils, such as Cremophor EL, vitamin E tocopherol propylene glycol succinate (Vitamin E TGPS); polyoxyethylene-polyoxypropylene block copolymers; poloxamers; and/or mixtures thereof. In an embodiment, the surfactant is sodium laurel sulfate or sodium dodecyl sulfate.

As set forth above, in an embodiment a composition of the present invention may comprise a filler; a binder; a diluent; a glidant; a lubricant; disintegrant; and/or a similar ingredient that facilitates the granulation and tableting process. Suitable fillers; binders; diluents; glidants; lubricants; disintegrants; and/or similar ingredients that facilitate the granulation and tableting, include, but are not limited to: sucrose, lactose, microcrystalline cellulose, croscarmellose sodium, magnesium stearate, talc, colloidal silicon dioxide, gum acacia, cholesterol, tragacanth, stearic acid, gelatin, casein, lecithin (phosphatides), carboxymethylcellulose calcium, carboxymethylcellulose sodium, methylcellulose, hydroxyethylcellulose, hydroxypropylcellulose, hydroxypropylmethylcellulose phthalate, noncrystalline cellulose, cetostearyl alcohol, cetyl alcohol, cetyl esters wax, dextrates, dextrin, lactose, dextrose, glyceryl monooleate, glyceryl monostearate, glyceryl palmitostearate, polyoxyethylene alkyl ethers, polyethylene glycols, polyoxyethylene castor oil derivatives, polyoxyethylene stearates, polyvinyl alcohol, similar ingredients and/or mixtures thereof.

An embodiment of the present invention may include multiple ingredients from a plurality of component classes, e.g. surfactants, binders etc. An embodiment of the present invention may also comprise multiple ingredients from any single component class or plurality of component classes. For example, a composition comprising a filler may comprise sucrose and lactose, and other fillers. Further, certain ingredients may fall within multiple component classes.

In an embodiment, a composition of the present invention comprises:

20 to 90%, by weight, 3-biphenyl-4-yl-(2S)-[(4′-trifluoromethyl-biphenyl-4-carbonyl)-amino]-propionic acid;

up to 10%, by weight, a water soluble polymer;

up to 10%, by weight, a surfactant;

wherein at least one of the water soluble polymer or the surfactant are present in the composition. In a further embodiment, the remainder of the composition may comprise a filler; a binder; a diluent; a glidant; a lubricant; a disintegrant, or a mixture thereof.

In another embodiment, a composition of the present invention comprises:

20 to 90%, by weight, 3-biphenyl-4-yl-(2S)-[(4′-trifluoromethyl-biphenyl-4-carbonyl)-amino]-propionic acid;

up to 10%, by weight, a water soluble polymer;

0.5 to 10%, by weight, a surfactant;

In a further embodiment, the remainder of the composition may comprise a filler; a binder; a diluent; a glidant; a lubricant; a disintegrant, or a mixture thereof.

In another embodiment, a composition of the present invention comprises:

20 to 90%, by weight, 3-biphenyl-4-yl-(2S)-[(4′-trifluoromethyl-biphenyl-4-carbonyl)-amino]-propionic acid;

0.5 to 10%, by weight, a water soluble polymer;

up to 10%, by weight, a surfactant;

In a further embodiment, the remainder of the composition may comprise a filler; a binder; a diluent; a glidant; a lubricant; a disintegrant, or a mixture thereof.

In another embodiment, a composition of the present invention comprises:

20 to 90%, by weight, 3-biphenyl-4-yl-(2S)-[(4′-trifluoromethyl-biphenyl-4-carbonyl)-amino]-propionic acid;

0.5 to 10%, by weight, a water soluble polymer;

0.5 to 10%, by weight, a surfactant.

In a further embodiment, the remainder of the composition may comprise a filler; a binder; a diluent; a glidant; a lubricant; a disintegrant, or a mixture thereof.

A composition of the present invention may comprise different physical forms, including, but not limited to: a tablet, a pill, or a capsule. The physical form, e.g. tablet, may comprise a desired dosage amount, by weight, of 3-biphenyl-4-yl-(2S)-[(4′-trifluoromethyl-biphenyl-4-carbonyl)-amino]-propionic acid. In an embodiment, a dosage amount by weight comprises approximately one-half of the total weight of the solid dosage form, for example 100 milligrams (mg) in a 200 mg tablet.

The preparation of 3-biphenyl-4-yl-(2S)-[(4′-trifluoromethyl-biphenyl-4-carbonyl)-amino]-propionic acid is described in US Patent Publication No. 20040110832, published Jun. 10, 2004, which is incorporated fully herein by reference above.

3-Biphenyl-4-yl-(2S)-[(4′-trifluoromethyl-biphenyl-4-carbonyl)-amino]-propionic acid may also be prepared by the following Examples A or B in the Examples section below.

A composition of the present invention may be prepared by a granulation process, and/or other processes generally utilized in the pharmaceutical arts. A composition of the present invention may be prepared by a method of the present invention, but may also be prepared by other methods.

In an embodiment of the present invention wherein the composition comprises a water soluble polymer and a surfactant, the composition can be prepared by a wet granulation process wherein 3-biphenyl-4-yl-(2S)-[(4′-trifluoromethyl-biphenyl-4-carbonyl)-amino]-propionic acid, a water soluble polymer, a surfactant, water, and optionally other excipients, such as a binder and/or a glidant, are mixed to form a wet granulation in a suitable granulator/mixer. The water may be added as part of an aqueous solution of a water soluble polymer and/or surfactant. The wet granulation is then dried and milled using a suitable milling device. The wet granulation and drying can be performed in a fluid bed granulator/dryer. The wet granulation can also be dried using a tray drying oven. The dried granulation may further be blended with a filler, lubricant and/or disintegrant before compression into tablets. The blending may be performed using a blender. The resulting composition of the present invention may be compressed into tablets, or other dosage forms. Alternatively, the solids may be filled in hard gelatin capsules. Other sequences of addition are possible and permissible. Further, the present invention also provides a dry formulation process.

As described above, in another aspect, the present invention provides methods and/or processes for producing compositions comprising 3-biphenyl-4-yl-(2S)-[(4′-trifluoromethyl-biphenyl-4-carbonyl)-amino]-propionic acid.

In an embodiment, a method of the present invention comprises:

mixing 3-biphenyl-4-yl-(2S)-[(4′-trifluoromethyl-biphenyl-4-carbonyl)-amino]-propionic acid, a water soluble polymer, water, and optionally other excipients;

granulating the mixture until a substantially uniform granulation is achieved;

drying the resulting granulation;

milling the dried granulations to a desired particle size; and

compressing the milled granulation into a desired physical form.

The method may further comprise blending the dried granulation, and/or dried and milled granulation, with a filler and/or disintegrant before compression into tablets.

In an embodiment, a method of the present invention comprises:

mixing 3-biphenyl-4-yl-(2S)-[(4′-trifluoromethyl-biphenyl-4-carbonyl)-amino]-propionic acid, a surfactant, and optionally other excipients.

The method may further comprise blending the dry formulation with a lubricant before packaging into a solid dosage form such as filling a capsule or compressing into a tablet.

In another embodiment, a method of the present invention comprises:

mixing 3-biphenyl-4-yl-(2S)-[(4′-trifluoromethyl-biphenyl-4-carbonyl)-amino]-propionic acid, a water soluble polymer, a surfactant, water, and optionally other excipients;

granulating the mixture until a substantially uniform granulation is achieved;

drying the resulting granulation;

milling the dried granulations to a desired particle size; and

compressing the milled granulation into a desired physical form.

The method may further comprise blending the dried granulation, and/or dried and milled granulation, with a filler and/or disintegrant before compression into tablets.

In a further embodiment, a method of the present invention comprises:

mixing 3-biphenyl-4-yl-(2S)-[(4′-trifluoromethyl-biphenyl-4-carbonyl)-amino]-propionic acid, a water soluble polymer, a surfactant, water, and optionally other excipients;

granulating the mixture until a substantially uniform granulation is achieved;

drying the resulting granulation;

milling the dried granulations to a desired particle size;

blending the dried and milled granulation, with a filler and/or disintegrant; and

compressing the milled granulation into a desired physical form.

In embodiments of a wet granulation method of the present invention, the drying step may be performed until the resulting granulation has a moisture content sufficient to permit further processing. In one non-limiting example, the moisture content is less than or equal to 5%, by weight. Milling may be performed until the resulting dried granulation is of sufficient size to flow freely and/or permit compaction in a tablet maker, such as when the particles of the dried granulation pass a 30 mesh screen.

Suitable water soluble polymers, surfactants, fillers, binders, diluents, glidants, lubricants, and/or disintegrants include those set forth above with reference to a composition of the present invention. The order of addition/combination of the ingredients may be varied.

A method of preparation of a formulation of the present invention may be performed utilizing apparatus known to those of ordinary skill in the art, including but not limited to, mixers, driers, granulators, fluid bed granulators, fluid bed driers, milling devices, blenders, containers, vessels and the like.

In a further aspect, the present invention provides methods for using compositions comprising 3-biphenyl-4-yl-(2S)-[(4′-trifluoromethyl-biphenyl-4-carbonyl)-amino]-propionic acid.

In an embodiment, a method for using compositions comprising 3-biphenyl-4-yl-(2S)-[(4′-trifluoromethyl-biphenyl-4-carbonyl)-amino]-propionic acid comprises ingesting a composition of the present invention.

In another embodiment a method for using compositions comprising 3-biphenyl-4-yl-(2S)-[(4′-trifluoromethyl-biphenyl-4-carbonyl)-amino]-propionic acid comprises providing a composition of the present invention to a patient suffering from a cardiovascular disease. The method may further comprise having the patient ingest the composition of the present invention.

In a further embodiment a method for using compositions comprising 3-biphenyl-4-yl-(2S)-[(4′-trifluoromethyl-biphenyl-4-carbonyl)-amino]-propionic acid comprises providing a composition of the present invention to a patient at risk for a cardiovascular disease. The method may further comprise having the patient ingest the composition of the present invention.

Further methods for using a composition of the present invention may be determined by one of ordinary skill in the art from the disclosure contained herein and provided in US Patent Publication No. 20040110832, published Jun. 10, 2004.

In another embodiment of the present invention, there is provided a pharmaceutical composition further comprising a therapeutically effective amount of 3-biphenyl-4-yl-(2S)-[(4′-trifluoromethyl-biphenyl-4-carbonyl)-amino]-propionic acid, wherein said therapeutically effective amount comprises a sufficient amount of the compound to at least partially inhibit the biological activity of factor IX in a subject, a sufficient amount of the compound for at least partial amelioration of at least one factor IX-mediated disease, or a sufficient amount of the compound to at least partially inhibit the intrinsic clotting cascade in a subject. In an embodiment of the pharmaceutical composition, said factor IX-mediated disease comprises stroke. In another embodiment of the pharmaceutical composition, said factor IX-mediated disease comprises deep vein thrombosis. In another embodiment of the pharmaceutical composition, said factor IX-mediated disease comprises deep vein thrombosis, wherein said thrombosis is associated with surgical procedures, long periods of confinement, acquired or inherited pro-coagulant states including anti-phospholipid antibody syndrome, protein C deficiency and protein S deficiency, or acute and chronic inflammation including recurrent miscarriage or Systemic Lupus Erythmatosis (SLE). In another embodiment, said factor IX-mediated disease comprises excessive clotting associated with the treatment of kidney diseases by hemodialysis and/or venous hemofiltration. In another embodiment, said factor IX-mediated disease comprises cardiovascular disease. In another embodiment, said factor IX-mediated disease comprises cardiovascular disease, wherein said cardiovascular disease comprises myocardial infarction, arrhythmia, or aneurysm. In another aspect, the present invention provides a pharmaceutical composition comprising a therapeutically effective amount of 3-biphenyl-4-yl-(2S)-[(4′-trifluoromethyl-biphenyl-4-carbonyl)-amino]-propionic acid, wherein said pharmaceutical composition is used to replace or supplement compounds that reduce clotting.

In another embodiment, the present invention provides a method for the inhibition of the normal biological function of factor IX comprising administering to a subject in need thereof a pharmaceutical composition of the present invention comprising 3-biphenyl-4-yl-(2S)-[(4′-trifluoromethyl-biphenyl-4-carbonyl)-amino]-propionic acid. In an embodiment of the method, said compound is an antagonist of factor IX activity. In another embodiment of the method, said compound antagonizes blood clotting mediated by factor IX.

In another embodiment, the present invention provides a method of treatment comprising administering to a subject in need thereof a pharmaceutical composition of the present invention comprising 3-biphenyl-4-yl-(2S)-[(4′-trifluoromethyl-biphenyl-4-carbonyl)-amino]-propionic acid, wherein said compound is administered in an amount sufficient to partially antagonize the biological activity of factor IX in said subject. In an embodiment, said amount sufficient to partially antagonize the biological activity of factor IX in a subject is an amount sufficient to achieve and maintain a sustained blood level that at least partially antagonizes factor IX. In a further embodiment, said sustained blood level is less than 1 μM. In another embodiment, said sustained blood level is greater than 0.1 μM.

In another embodiment, the present invention provides a method of treatment comprising administering to a subject in need thereof a pharmaceutical composition of the present invention comprising 3-biphenyl-4-yl-(2S)-[(4′-trifluoromethyl-biphenyl-4-carbonyl)-amino]-propionic acid, wherein said pharmaceutical composition is administered in the form of an oral dosage. In another embodiment of the method, the oral dosage is in tablet form. In another embodiment of the method, said compound is administered as a dose in a range from about 0.5 to 5 mg/kg of body weight per day. In another embodiment, said pharmaceutical composition is used to replace or supplement compounds that reduce clotting.

In another embodiment, the present invention provides a method for the inhibition of the normal biological function of factor IX comprising administering to a subject in need thereof a pharmaceutical composition of the present invention comprising a therapeutically effective amount of 3-biphenyl-4-yl-(2S)-[(4′-trifluoromethyl-biphenyl-4-carbonyl)-amino]-propionic acid, wherein said therapeutically effective amount of the compound comprises a sufficient amount of the compound for treatment of a factor IX-mediated disease.

In another aspect, the present invention provides a method of treatment of a disease or condition comprising administering to a subject in need thereof a pharmaceutical composition of the present invention comprising a therapeutically effective amount of 3-biphenyl-4-yl-(2S)-[(4′-trifluoromethyl-biphenyl-4-carbonyl)-amino]-propionic acid, wherein the disease or condition is selected from the group consisting of cardiovascular disease including myocardial infarction, arrhythmia, or aneurysm; stroke; deep vein thrombosis wherein the thrombosis may be associated with surgical procedures, long periods of confinement, acquired or inherited pro-coagulant states including anti-phospholipid antibody syndrome, protein C deficiency and protein S deficiency, or acute and chronic inflammation including recurrent miscarriage or Systemic Lupus Erythmatosis (SLE); clotting associated with the treatment of kidney disease by hemodialysis and/or venous hemofiltration.

Generally speaking, the compound pharmaceutical composition of the present invention is administered at a dosage level of less than 10 mg of compound/kg of body weight per day. In another embodiment, the dosage level of administration is greater than 0.5 mg of compound/kg of body weight per day. The amount of active ingredient that may be combined with the carrier materials to produce a single dosage will vary depending upon the host treated and the particular mode of administration. For example, in one non-limiting embodiment, a dosage unit forms, such as a tablet, intended for oral administration to humans may contain about 100 mg of 3-biphenyl-4-yl-(2S)-[(4′-trifluoromethyl-biphenyl-4-carbonyl)-amino]-propionic acid with an appropriate and convenient amount of carrier material which may vary up to about 20 percent of the total composition. The dosage may be individualized by the clinician based on the specific clinical condition of the subject being treated. Thus, it will be understood that the specific dosage level for any particular patient will depend upon a variety of factors including the activity of the specific compound employed, the age, body weight, general health, sex, diet, time of administration, route of administration, rate of excretion, drug combination and the severity of the particular disease undergoing therapy.

In another embodiment, the present invention provides a method to increase the bioavailability of 3-biphenyl-4-yl-(2S)-[(4′-trifluoromethyl-biphenyl-4-carbonyl)-amino]-propionic acid in a subject comprising administering to a subject a pharmaceutical composition comprising:

20 to 90%, by weight, 3-biphenyl-4-yl-(2S)-[(4′-trifluoromethyl-biphenyl-4-carbonyl)-amino]-propionic acid;

up to 10%, by weight, a water soluble polymer;

0.5 to 10%, by weight, a surfactant.

In a further embodiment, the remainder of the composition may comprise a filler; a binder; a diluent; a glidant; a lubricant; a disintegrant, or a mixture thereof. In an embodiment, the surfactant comprises polysorbate 80, sodium lauryl sulfate, sodium dodecyl sulfate, salts of bile acids (taurocholate, glycocholate, cholate, deoxycholate, etc.) which may be combined with lecithin; ethoxylated vegetable oils, such as Cremophor EL, vitamin E tocopherol propylene glycol succinate (Vitamin E TGPS); polyoxyethylene-polyoxypropylene block copolymers; poloxamers; and/or mixtures thereof. In another embodiment, the surfactant comprises sodium lauryl sulfate or sodium dodecyl sulfate. In another embodiment, the pharmaceutical composition is administered in the form of an oral dosage. In another embodiment, the pharmaceutical composition is in tablet form.

Embodiments of the present invention are further illustrated by the following examples.

### EXAMPLES

### Example A

### Preparation of 3-Biphenyl-4-yl-(2S)-[(4′-trifluoromethyl-biphenyl-4-carbonyl)-amino]-propionic Acid

(2S)-Amino-3-biphenyl-4-yl-propionic acid methyl ester (1.0-1.5 eq) was reacted with 4-bromo-benzoic acid (1.0-1.5 eq) in a solution of dimethylformamide (DMF), o-benzotriazolyl-N,N,N′,N′-tetramethyluronium hexafluorophosphate (HBTU) (1.0-1.5 eq), and diisopropylethylamine (DIEA) (2.0-3.0 eq). After completion of the reaction, a sufficient amount of water was added, and the mixture was extracted with ethyl acetate. The combined organic layers were washed with water and brine, and then dried over sodium sulfate. The solvent was removed in vacuo to afford 3-biphenyl-4-yl-(2S)-[(5-bromo-benzoyl)-amino]-propionic acid methyl ester, which was either used without further purification or purified by flash chromatography.

3-Biphenyl-4-yl-(2S)-[(5-bromo-benzoyl)-amino]-propionic acid methyl ester (1.0 eq) was reacted with 4-trifluoromethyl phenyl boronic acid (3.0 eq) in 1,2 dimethoxyethane (DME) or toluene using palladium tetrakis-triphenylphosphine (Pd(PPh₃)₄) (0.05 eq), 2N Na₂CO₃ solution. The mixture was heated at 75° C. for 12 h. After completion of the reaction, solvent was evaporated in vacuo. During the reaction, some of the ester may be hydrolyzed to the corresponding acid. Therefore, crude product so obtained may be re-esterified by dissolving it in methanol containing 1% HCl and refluxing. After the completion of the reaction, the mixture was concentrated in vacuo, and the residue was purified by column chromatography to provide 3-biphenyl-4-yl-(2S)-[(4′-trifluoromethyl-biphenyl-4-carbonyl)-amino]-propionic acid methyl ester.

The ester was hydrolyzed in tetrahydrofuran/methanol (4:1) and 2N-lithium hydroxide solution (5 eq) was added. The mixture was stirred at 0° C. and then warmed to room temperature. After the reaction was complete, 2N HCl added to neutralize the mixture, and the mixture was extracted with ethyl acetate. The organic layer was washed with brine, dried over sodium sulfate, and the solvent was removed in vacuo to afford 3-biphenyl-4-yl-(2S)-[(4′-trifluoromethyl-biphenyl-4-carbonyl)-amino]-propionic acid.

### Example B

### Preparation of 3-Biphenyl-4-yl-(2S)-[(4′-trifluoromethyl-biphenyl-4-carbonyl)-amino]-propionic Acid

Step A—4′-trifluoromethyl-biphenyl-4-carboxylic Acid

To a solution of water/acetonitrile (1:1), Na₂CO₃ (2.0 eq), 4-carboxybenzene boronic acid (1.0 eq) was added 4-bromobenzotrifluoride (1.2 eq) and Pd(PPh₃)₄ (0.05 eq). The reaction was heated to about 76° C. until the reaction was complete as evidenced by HPLC.

After the reaction was complete, the reaction mixture was cooled and filtered through a celite pad. The pad was washed successively with a solution of water/acetonitrile (1:1). The filtrate was cooled on an ice bath, and 3N HCl was slowly added until the filtrate reached a pH of about 2. The solid precipitate that formed was filtered and washed successively with water and diethyl ether. The resulting 4′-trifluoromethyl-biphenyl-4-carboxylic acid was dried in vacuo at 45° C. and used without further purification.

Step B—4,4′-Biphenylalanine Methyl Ester Hydrochloride

To a solution of L-4,4′-biphenyl alanine (1.0 eq) in anhydrous methanol was slowly added thionyl chloride (SOCl₂) (1.5 eq) at ambient temperature. After the addition of SOCl₂ was complete, the mixture was refluxed until the reaction was complete as evidenced by HPLC.

When complete, the mixture was concentrated in vacuo using a water bath (45-50° C.). The solid residue was taken up in methanol/toluene (1:1) and concentrated in vacuo using a water bath (45-50° C.). The resulting 4,4′-biphenylalanine methyl ester hydrochloride was dried in vacuo at 45° C. and used without further purification.

Step C—3-Biphenyl-4-yl-(2S)-[(4′-trifluoromethyl-biphenyl-4-carbonyl)-amino]-propionic Acid Methyl Ester

To a solution of 4′-trifluoromethyl-biphenyl-4-carboxylic acid (1.0 eq) in methylene chloride/DMF (20:1) at ambient temperature was slowly added SOCl₂ (1.5 eq). After addition was complete, the mixture was refluxed until the reaction was complete as evidenced by ¹H NMR. Upon completion, the mixture was filtered, and the filtrate was concentrated in vacuo using a water bath (45-50° C.). The resulting 4′-trifluoromethyl-biphenyl-4-carboxylic acid chloride was taken up in toluene and the mixture was concentrated to dryness in vacuo using a water bath (45-50° C.).

To an ambient solution of 4′-trifluoromethyl-biphenyl-4-carboxylic acid chloride (1.0 eq) and 4,4′-biphenylalanine methyl ester hydrochloride (1.1 eq) in 1,4-dioxane was slowly added DIEA (2.25 eq). After addition was complete, the reaction was followed by HPLC until complete. Upon completion, the mixture was neutralized by the addition of 2N HCl and concentrated in vacuo using a water bath (45-50° C.) until a precipitate formed. Cold water (ca. 5° C.) was added to the heterogenous mixture, and the mixture was filtered. The collected residue was washed with cold water, air dried, washed with methanol, and air dried. The resulting 3-biphenyl-4-yl-(2S)-[(4′-trifluoromethyl-biphenyl-4-carbonyl)-amino]-propionic acid methyl ester was dried in vacuo at 45° C. and used without further purification.

Step D—3-Biphenyl-4-yl-(2S)-[(4′-trifluoromethyl-biphenyl-4-carbonyl)-amino]-propionic Acid

To a solution of 3-biphenyl-4-yl-(2S)-[(4′-trifluoromethyl-biphenyl-4-carbonyl)-amino]-propionic acid methyl ester (1.0 eq) in DMF/THF (1:4) was slowly added aqueous LiOH (5.0 eq). The reaction was followed by HPLC. Upon completion, the mixture was cooled, and 5 M HCl was added until the mixture had a pH of about 2. The mixture was concentrated in vacuo to remove the organic solvents. The concentrated mixture was diluted with water and a precipitate formed upon cooling the mixture on an ice-bath. The precipitate was filtered and washed with water until the washings were neutral. The resulting 3-biphenyl-4-yl-(2S)-[(4′-trifluoromethyl-biphenyl-4-carbonyl)-amino]-propionic acid was air dried and then dried under vacuum. The product may be further purified by recrystallizing in methanol or the product may be used without further purification.

### Example 1

### Wet Granulation of 3-Biphenyl-4-yl-(2S)-[(4′-trifluoromethyl-biphenyl-4-carbonyl)-amino]-propionic Acid

The following procedure was used to prepare a tablet containing 100 mg of 3-biphenyl-4-yl-(2S)-[(4′-trifluoromethyl-biphenyl-4-carbonyl)-amino]-propionic acid containing the ingredients listed in Table 1. The amounts listed in Table 1 represent amounts used to prepare a 5 kg batch.

Microcrystalline cellulose, croscarmellose sodium, lactose monohydrate, colloidal silicone dioxide were screened through a 20 mesh screen, and transferred into a high shear mixer and mixed at low speed (about 200 rpm). 3-Biphenyl-4-yl-(2S)-[(4′-trifluoromethyl-biphenyl-4-carbonyl)-amino]-propionic acid was screened through a 16 mesh screen and transferred into the high shear mixer. The mixture was mixed at low speed (ca. 5 min), passed through a comill using a 032R screen, and transferred back into the high shear mixer.

Sodium lauryl sulfate (SLS) and povidone (PVP) were dissolved in sufficient quantity of water to achieve solution. The SLS/PVP solution was used to granulate the mixture of powders in the high shear mixer. While the SLS/PVP solution was added to the mixture, the mixture was mixed at low speed for several minutes with the chopper off. After the addition of the SLS/PVP solution was complete, the mixture was mixed at high speed (over 1500 rpm) for several minutes. Additional amounts of water may be added during mixing if the granulation was too dry as evidenced by ability of granulation to clump.

The granulation was mixed until uniformity was achieved. The granulation was dried in a fluid bed dryer such that the Loss-on-Drying (105° C.) of the dried formulation was within a target range. In this example, the LOD was between 1.5 to 2.5% by weight, however, an optimal LOD may vary depending upon the batch size and other factors. The particle size of the dried granulation was controlled by passing the mixture through a 30-mesh screen. Any oversized granulation was passed through a 30 mesh screen using a Quadro comill with 039R screen.

The dried granulation and additional quantities of microcrystalline cellulose and croscarmellose sodium were added to a V-blender and mixed (ca. 15 min). A portion of the mixture was removed from the V-blender and blended with magnesium stearate. The portion was added back to the V-blender and mixed with the original mixture for several minutes.

The resulting mixture was compressed into tablets having about 100 mg of 3-Biphenyl-4-yl-(2S)-[(4′-trifluoromethyl-biphenyl-4-carbonyl)-amino]-propionic acid. Compression was carried out on a rotary tablet press using appropriate size and tooling.

TABLE 1
Ingredients for wet granulation tablet (100 mg)

| Ingredient | Amount (g) |
|---|---|
| 3-Biphenyl-4-yl-(2S)-[(4′-trifluoromethyl- | 2,500.0 |
| biphenyl-4-carbonyl)-amino]-propionic acid ^{a} | |
| Microcrystalline cellulose (Avicel PH 101) ^{b}, NF, | 1,225.0 |
| Ph. Eur. | |
| Lactose monohydrate (Fast Flo), NF, Ph. Eur. | 450.0 |
| Croscarmellose sodium (Ac-di-sol) ^{b}, NF, Ph. Eur. | 525.0 |
| Colloidal silicon dioxide (Cabosil M5P), NF, Ph. Eur. | 25.0 |
| Povidone (Kollidon 30), USP, Ph. Eur. | 150.0 |
| Sodium lauryl sulfate, NF, Ph. Eur. | 100.0 |
| Purified water ^{c} | |
| Magnesium stearate, NF, Ph. Eur. | 25.0 |
| Total | 5000.0 |

^{a} The quantity of 3-biphenyl-4-yl-(2S)-[(4′-trifluoromethyl-biphenyl-4-carbonyl)-amino]-propionic acid is adjusted based on the drug substance potency and chiral purity (% R-isomer). A corresponding reduced amount of lactose will be adjusted to maintain the same tablet weight.
^{b} Microcrystalline cellulose and croscarmellose sodium are added as intragranular and extragranular excipients.
^{c} Removed during processing

### Example 2

### Dry Formulation of 3-Biphenyl-4-yl-(2S)-[(4′-trifluoromethyl-biphenyl-4-carbonyl)-amino]-propionic Acid

The following procedure was used to prepare a tablet containing 100 mg of 3-biphenyl-4-yl-(2S)-[(4′-trifluoromethyl-biphenyl-4-carbonyl)-amino]-propionic acid containing the ingredients listed in Table 2. The amounts listed in Table 2 represent amounts used to prepare a 0.976 kg batch.

Talc (screened through 20-mesh screen), 3-Biphenyl-4-yl-(2S)-[(4′-trifluoromethyl-biphenyl-4-carbonyl)-amino]-propionic acid, colloidal silicon dioxide, sodium lauryl sulfate, and sodium starch glycolate were added to a V-shell blender and mixed. The resulting mixture was screened through a 20-mesh screen and returned to the V-shell blender. Pre-gelatinized starch and microcrystalline cellulose (screened through a 20-mesh screen) were added to the mixture in the V-shell blender and mixed. A portion of the resulting mixture was removed from the V-shell blender. The portion was mixed with the magnesium stearate, passed through a 20-mesh screen, and returned to the V-shell blender. The final mixture was blended prior to capsule filling. The capsules (hard gelatin, white, opaque, size 0) were filled to the appropriate theoretical capsule weight.

TABLE 2
Ingredients for dry formulation capsule (100 mg)

| Ingredient | Amount (g) |
|---|---|
| 3-Biphenyl-4-yl-(2S)-[(4′-trifluoromethyl- | 450.0 |
| biphenyl-4-carbonyl)-amino]-propionic acid¹ | |
| Pregelatinized Starch, NF | 292.5 |
| Microcrystalline Cellulose (Avicel PH 102), NF | 45.0 |
| Sodium Starch Glycolate, NF | 99.0 |
| Colloidal Silicon Dioxide, NF | 22.5 |
| Talc, USP | 54.0 |
| Magnesium Stearate NF | 9.0 |
| Sodium Lauryl Sulfate, NF | 4.5 |
| Total | 976.5 |
| Capsule shell, hard gelatin, white, opaque, Size 0 | |

Note:
¹The quantity of 3-Biphenyl-4-yl-(2S)-[(4′-trifluoromethyl-biphenyl-4-carbonyl)-amino]-propionic acid may be adjusted based on the drug substance lot factor, which is calculated to reflect the purity along with the water and residual solvents content. A corresponding reduced amount of pregelatinized starch will be adjusted to maintain the same fill weight per capsule.

The compositions of the dry blend capsules of Example 2 and the wet formulation tablets of Example 1 are summarized in Table 3.

TABLE 3
Composition of Dry Formulation Capsules
and Wet Granulation Tablets.

| | | Example | Example |
|---|---|---|---|
| Ingredient | Function | 1 % w/w | 2 % w/w |
| 3-Biphenyl-4-yl-(2S)-[(4′- | Active | 50.0 | 46.1 |
| trifluoromemyl-biphenyl-4- | Ingredient | | |
| carbonyl)-amino]-propionic | | | |
| acid | | | |
| Pregelatinized Starch NF | Water | | 30.0 |
| | Insoluble | | |
| | Diluent | | |
| Microcrystalline Cellulose | Water | 24.5 | |
| (Avicel PH 101) NF, Ph. Eur. | Insoluble | | |
| | Diluent | | |
| Microcrystalline Cellulose | Water | | 4.6 |
| (Avicel PH 102) NF, Ph. Eur. | Insoluble | | |
| | Diluent | | |
| Sodium Starch Glycolate NF | Disintegrant | | 10.1 |
| Croscarmellose sodium | Disintegrant | 10.5 | |
| (Ac-di-sol) NF, Ph. Eur. | | | |
| Colloidal Silicon Dioxide | Glidant | 0.5 | 2.3 |
| (Cabosil M5P) NF, Ph. Eur. | | | |
| Talc USP | Glidant | | 5.5 |
| Magnesium Stearate NF, | Lubricant | 0.5 | 0.9 |
| Ph. Eur. | | | |
| Lactose monohydrate | Water | 9.0 | |
| (Fast Flo) NF, Ph. Eur. | Soluble | | |
| | Diluent | | |
| Sodium Lauryl Sulfate | Surfactant | 2.0 | 0.5 |
| NF, Ph. Eur. | | | |
| Povidone (Kollidon 30), | Binder | 3.0 | |
| USP, Ph. Eur. | | | |
| Pure Water USP | | Qs | |
| Fill weight | | 100% | 100% |
| Capsule shell, hard gelatin, | | | 1 per |
| white, opaque, Size 0 | | | capsule |

### Example 3

Dissolution of the wet granulation tablets of Example 1 and the dry formulation capsules of Example 2 was determined using USP method 2 (paddles at 75 rpm, 0.025 M NaH₂PO₄ with 2% sodium dodecyl sulfate (SDS), pH 6.8). Table 4 shows a comparison of the dissolution in the NaH₂PO₄/SDS solution of (a) micronized 3-biphenyl-4-yl-(2S)-[(4′-trifluoromethyl-biphenyl-4-carbonyl)-amino]-propionic acid (2×100 mg), (b) a wet granulation tablet of Example 1 (2×100 mg), and (c) a dry blend capsules of Example 2 (2×100 mg). The results show that the wet granulation using the water soluble polymer Povidone and the surfactant sodium laurel sulfate enhanced dissolution of 3-biphenyl-4-yl-(2S)-[(4′-trifluoromethyl-biphenyl-4-carbonyl)-amino]-propionic acid, and thereby provide enhanced bioavailability relative to the dry blend capsules of Example 2.

TABLE 4

| | Percent 3-Biphenyl-4-yl-(2S)-[(4′-trifluoromethyl- | | |
|---|---|---|---|
| | biphenyl-4-carbonyl)-amino]-propionic acid Dissolved | | |
| | Comparative | Example 1 | Example 2 |
| Time | (Micronized drug | (Wet Granulation | (Dry Blend |
| (min) | substance) | Tablets) | Capsules) |
| 0 | 0 | 0 | 0 |
| 15 | 43.2 | 98.8 | 81.5 |
| 30 | 57.0 | 100.8 | 92.5 |
| 45 | 67.3 | 100.6 | 96.5 |
| 60 | 73.9 | 101 | 98.7 |

While the invention has been described and illustrated with reference to certain preferred embodiments thereof, those skilled in the art will appreciate that various changes, modifications and substitutions can be made therein without departing from the spirit and scope of the invention. For example, effective dosages other than the preferred dosages as set forth herein may be applicable as a consequence of variations in the responsiveness of the mammal being treated for factor IXa-mediated disease(s). Likewise, the specific pharmacological responses observed may vary according to and depending on the particular active compound selected or whether there are present pharmaceutical carriers, as well as the type of formulation and mode of administration employed, and such expected variations or differences in the results are contemplated in accordance with the objects and practices of the present invention.

What is claimed is:

1. A composition comprising 3-biphenyl-4-yl-(2S)-[(4′-trifluoromethyl-biphenyl-4-carbonyl)-amino]-propionic acid and a water soluble polymer.

2. The composition of claim 1 further comprising a surfactant.

3. The composition of claim 1, wherein the water soluble polymer is selected from the group consisting of PVP, hydroxypropylmethylcellulose, polyethylene glycol, cyclodextrin, and mixtures thereof.

4. The composition of claim 1, wherein the water soluble polymer is PVP.

5. The composition of claim 1, wherein the water soluble polymer is present in an amount greater than 0.5 wt %.

6. The composition of claim 2, wherein the surfactant is selected from the group consisting of polysorbate 80, sodium lauryl sulfate, sodium dodecyl sulfate, a salt of a bile acid, an ethoxylated vegetable oil, a polyoxyethylene-polyoxypropylene block copolymer, a poloxamer and/or mixtures thereof.

7. The composition of claim 2, wherein the surfactant is sodium lauryl sulfate or sodium dodecyl sulfate.

8. A composition comprising 3-biphenyl-4-yl-(2S)-[(4′-trifluoromethyl-biphenyl-4-carbonyl)-amino]-propionic acid and a surfactant.

9. The composition of claim 8, wherein the surfactant is selected from the group consisting of polysorbate 80, sodium lauryl sulfate, sodium dodecyl sulfate, a salt of a bile acid, an ethoxylated vegetable oil, a polyoxyethylene-polyoxypropylene block copolymer, a poloxamer and/or mixtures thereof.

10. The composition of claim 9, wherein the surfactant is sodium lauryl sulfate or sodium dodecyl sulfate.

11. The composition of claim 8 further comprising a water soluble polymer.

12. The composition of claim 11, wherein the water soluble polymer is selected from the group consisting of PVP, hydroxypropylmethylcellulose, polyethylene glycol, cyclodextrin, and/or mixtures thereof.

13. The composition of claim 12, wherein the water soluble polymer is PVP.

14. The composition of claim 11, wherein the water soluble polymer is present in an amount greater than 0.5 wt %.

15. A composition comprising
a) a pharmaceutically effective amount of 3-biphenyl-4-yl-(2S)-[(4′-trifluoromethyl-biphenyl-4-carbonyl)-amino]-propionic acid or a pharmaceutically acceptable salt thereof;
b) up to 10%, by weight, a water soluble polymer;
c) up to 10%, by weight a surfactant;
wherein at least one of the water soluble polymer or the surfactant are present in the composition.

16. A composition comprising 3-biphenyl-4-yl-(2S)-[(4′-trifluoromethyl-biphenyl-4-carbonyl)-amino]-propionic acid prepared by wet granulation process.

17. A method for preparing a composition comprising
mixing 3-biphenyl-4-yl-(2S)-[(4′-trifluoromethyl-biphenyl-4-carbonyl)-amino]-propionic acid, a water soluble polymer, water, and optionally other excipients;
granulating the mixture until a substantially uniform granulation is achieved;
drying the resulting granulation;
milling the dried granulations to a desired particle size; and
compressing the milled granulation into a desired physical form.

18. The method of claim 17 further comprising blending the dried and milled granulation, with a binder, filler and/or disintegrant before compression into tablets.

19. The method of claim 17 further comprising mixing a surfactant with the 3-biphenyl-4-yl-(2S)-[(4′-trifluoromethyl-biphenyl-4-carbonyl)-amino]-propionic acid and the water soluble polymer.

20. A method for preparing composition comprising
mixing 3-biphenyl-4-yl-(2S)-[(4′-trifluoromethyl-biphenyl-4-carbonyl)-amino]-propionic acid, a surfactant, and optionally other excipients.

21. The method of claim 20 further comprising blending the dry formulation with a lubricant.

22. The method of claim 21, wherein the dry formulation is filled into a capsule.

23. A method for using a composition comprising 3-biphenyl-4-yl-(2S)-[(4′-trifluoromethyl-biphenyl-4-carbonyl)-amino]-propionic acid the method comprising ingesting a composition of claim 1.

24. A method for using a composition comprising 3-biphenyl-4-yl-(2S)-[(4′-trifluoromethyl-biphenyl-4-carbonyl)-amino]-propionic acid the method comprising providing a composition of claim 1 to a patient suffering from a cardiovascular disease.

25. The method of claim 24 further comprising having the patient ingest a composition comprising 3-biphenyl-4-yl-(2S)-[(4′-trifluoromethyl-biphenyl-4-carbonyl)-amino]-propionic acid and a water soluble polymer.

26. A method for using a composition comprising 3-biphenyl-4-yl-(2S)-[(4′-trifluoromethyl-biphenyl-4-carbonyl)-amino]-propionic acid the method comprising providing a composition of claim 1 to an individual at risk for a cardiovascular disease.

27. The method of claim 26 further comprising having the patient ingest the composition of claim 1.

28. A method for synthesizing 3-biphenyl-4-yl-(2S)-[(4′-trifluoromethyl-biphenyl-4-carbonyl)-amino]-propionic acid comprising:
a) adding 4-carboxybenzene boronic acid to 4-bromobenzotrifluoride and Pd(PPh₃)₄ to generate 4′-trifluoromethyl-biphenyl-4-carboxylic acid;
b) adding thionyl chloride to L-4,4′-biphenyl alanine in methanol to generate (2S)-amino-3-biphenyl-4-yl-propionic acid methyl ester;
c) adding thionyl chloride to 4′-trifluoromethyl-biphenyl-4-carboxylic acid to generate 4′-trifluoromethyl-biphenyl-4-carboxylic acid chloride;
d) reacting (2S)-amino-3-biphenyl-4-yl-propionic acid methyl ester with 4′-trifluoromethyl-biphenyl-4-carboxylic acid chloride to generate 3-biphenyl-4-yl-(2S)-[(4′-trifluoromethyl-biphenyl-4-carbonyl)-amino]-propionic acid methyl ester;
e) hydrolyzing 3-biphenyl-4-yl-(2S)-[(4′-trifluoromethyl-biphenyl-4-carbonyl)-amino]-propionic acid methyl ester to generate 3-biphenyl-4-yl-(2S)-[(4′-trifluoromethyl-biphenyl-4-carbonyl)-amino]-propionic acid.

29. A pharmaceutical composition of claim 15, further comprising a therapeutically effective amount of 3-biphenyl-4-yl-(2S)-[(4′-trifluoromethyl-biphenyl-4-carbonyl)-amino]-propionic acid, wherein said therapeutically effective amount comprises a sufficient amount of the compound to at least partially inhibit the biological activity of factor IX in a subject, a sufficient amount of the compound for at least partial amelioration of at least one factor IX-mediated disease, or a sufficient amount of the compound to at least partially inhibit the intrinsic clotting cascade in a subject.

30. A method for the inhibition of the normal biological function of factor IX comprising administering to a subject in need thereof a pharmaceutical composition of claim 15.

31. A method of treatment comprising administering to a subject in need thereof a pharmaceutical composition of claim 15 comprising 3-biphenyl-4-yl-(2S)-[(4′-trifluoromethyl-biphenyl-4-carbonyl)-amino]-propionic acid, wherein said compound is administered in an amount sufficient to partially antagonize the biological activity of factor IX in said subject.

32. The method of claim 31, wherein said amount sufficient to partially antagonize the biological activity of factor IX in a subject is an amount sufficient to achieve and maintain a sustained blood level that at least partially antagonizes factor IX.

33. The method of claim 32, wherein said sustained blood level is less than 1 μM.

34. The method of claim 32, wherein said sustained blood level is greater than 0.1 μM.

35. A method of treatment comprising administering to a subject in need thereof a pharmaceutical composition of claim 15 comprising 3-biphenyl-4-yl-(2S)-[(4′-trifluoromethyl-biphenyl-4-carbonyl)-amino]-propionic acid, wherein said pharmaceutical composition is administered in the form of an oral dosage.

36. The method of claim 35, wherein the oral dosage is in tablet form.

37. The method of claim 35, wherein said compound is administered as a dose in a range from about 0.5 to 5 mg/kg of body weight per day.

38. A method for the inhibition of the normal biological function of factor IX comprising administering to a subject in need thereof a pharmaceutical composition of claim 15 comprising a therapeutically effective amount of 3-biphenyl-4-yl-(2S)-[(4′-trifluoromethyl-biphenyl-4-carbonyl)-amino]-propionic acid, wherein said therapeutically effective amount of the compound comprises a sufficient amount of the compound for treatment of a factor IX-mediated disease.

39. A method of treatment of a disease or condition comprising administering to a subject in need thereof a pharmaceutical composition of the present invention comprising a therapeutically effective amount of 3-biphenyl-4-yl-(2S)-[(4′-trifluoromethyl-biphenyl-4-carbonyl)-amino]-propionic acid, wherein the disease or condition is selected from the group consisting of cardiovascular disease including myocardial infarction, arrhythmia, or aneurysm; stroke; deep vein thrombosis wherein the thrombosis may be associated with surgical procedures, long periods of confinement, acquired or inherited pro-coagulant states including anti-phospholipid antibody syndrome, protein C deficiency and protein S deficiency, or acute and chronic inflammation including recurrent miscarriage or Systemic Lupus Erythmatosis (SLE); clotting associated with the treatment of kidney disease by hemodialysis and/or venous hemofiltration.

40. A method to increase the bioavailability of 3-biphenyl-4-yl-(2S)-[(4′-trifluoromethyl-biphenyl-4-carbonyl)-amino]-propionic acid in a subject comprising administering to a subject a pharmaceutical composition comprising:
20 to 90%, by weight, 3-biphenyl-4-yl-(2S)-[(4′-trifluoromethyl-biphenyl-4-carbonyl)-amino]-propionic acid;
up to 10%, by weight, a water soluble polymer; and
0.5 to 10%, by weight, a surfactant.

41. The method of claim 40, wherein the surfactant comprises polysorbate 80, sodium lauryl sulfate, sodium dodecyl sulfate, salts of bile acids (taurocholate, glycocholate, cholate, deoxycholate, etc.) which may be combined with lecithin; ethoxylated vegetable oils, such as Cremophor EL, vitamin E tocopherol propylene glycol succinate (Vitamin E TGPS); polyoxyethylene-polyoxypropylene block copolymers; poloxamers; and/or mixtures thereof.

42. The method of claim 40, wherein the surfactant comprises sodium lauryl sulfate or sodium dodecyl sulfate.

43. The method of claim 40, wherein the pharmaceutical composition is administered in the form of an oral dosage.

44. The method of claim 43, wherein the pharmaceutical composition is in tablet form.
